(19) United States
(12) Patent Application Publication
(10) Pub. No.: US 2009/0293347 A1
(43) Pub. Date: Dec. 3, 2009

(54) Method and apparatus for preparing an ethanol/water mixture

(75) Inventor: Antti Pasanen, Espoo (FI)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: St 1 Biofuels OY, Espoo (FI)

(21) Appl. No.: 11/920,784

(22) PCT Filed: May 24, 2006
(86) PCT No.: PCT/FI2006/000163

§ 371 Date: Jan. 24, 2008

(30) Foreign Application Priority Data
May 25, 2005 (FI) 20050554

(51) Int. Cl. C10L 1/18; C12P 7/06; C12M 1/00

(52) U.S. Cl. 44/437; 435/161; 435/289.1

(57) ABSTRACT

The invention relates to a method and apparatus for preparing an ethanol/water mixture, in which fermentable raw materials selected from sugars and raw materials capable of being hydrolyzed into fermentable sugars, and the necessary auxiliary substances are fed into a reactor; the raw materials are fermented in the reactor, ethanol/water mixture is separated from the reactor fermentation solution in an evaporator; and non-fermented matter is removed from the reactor. According to the invention, the reactor fermentation solution is fed continuously into a first falling-film evaporator during fermentation; the ethanol/water mixture is evaporated in the first falling-film evaporator, the evaporate obtained from the evaporator is condensed, and the condensed ethanol/water mixture is concentrated in a second falling-film evaporator, which is arranged in series with the first falling-film evaporator; water is removed as underflow from the second evaporator; and non-volatile matter is removed from the first evaporator and is recycled to the reactor.

### RELATED APPLICATIONS

This application is the U.S. National Stage of International Application No. PCT/FI2006/000163, filed May 24, 2006, published in English, and claims priority under 35 U.S.C. § 119 or 365 to Finland Application No. 20050554, filed May 25, 2005.

### BACKGROUND OF THE INVENTION

In modern societies, more and more efficient methods for treating waste and waste water are required of operations that produce waste and waste water. Waste waters and waste produced by industry and communities and containing various fermentable sugars or compounds capable of being converted into fermentable sugars can be treated by fermenting them into ethanol, which reduces the biological oxygen demand (BOD) and chemical oxygen demand (COD) loads contained in the waste waters of industry and communities, and reduces the amount of carbohydrates in waste. Ethanol prepared from waste and waste waters can be refined to suite various uses. The problem is that the waste and minor flows suited for preparing ethanol often are dispersed, whereby their transportation and costs associated with transportation create an obstacle to economical operations. Furthermore, it has been estimated that it is too expensive to invest in known processes for preparing ethanol from dispersed small flows with an output rate of 200-2,000 tons of ethanol/year.

The known industrially applied preparation processes of ethanol that are based on fermentation are relatively big in capacity, about 10,000-150,000 tons of ethanol/year. A part of the plants are based on batch fermentation and an ever increasing part in continuous fermentation. Fermented ethanol can be distilled to provide e.g. ethanol/water mixture of 95 percent by weight or thinner using a distillation apparatus. A distillation plant usually comprises stripping which is used to separate the mash from the ethanol/water mixture. Distillation is usually a one or multi-stage batch or continuous distillation. Evaporated distillate can be liquefied using condensers.

In big plants of the type described above, the non-fermented protein-, sugar- and yeast-containing solid matter as well as the ethanol-free mash or syrup after the stripping can be separated and used as animal fodder. In present industrial bioethanol production plants, as a result of fermentation, carbon dioxide is formed that can be separated from the gaseous phase flowing out of the fermenter using e.g. a gas meter. However, a problem with the present ethanol production plants is that sugar-containing, non-exploitable waste and waste waters with a relatively high COD value are produced. The mash does not always meet, for example, the quality requirements set by fodder suppliers, whereby it must be treated in some other manner, such as by burning or disposing of.

The problem with applying the present known preparation methods of ethanol to small-scale industrial production (less than 10,000 tons of ethanol per year) has been, among others: a) the complexity of the way of use and structure of the equipment necessary in the implementation of the methods, b) relatively high investment costs of the equipment and operating costs associated with the use of the equipment, c) the amount and quality of the waste and minor flows being created, d) the large amount of personal work required by the equipment, and d) legislation associated with flammable liquids, such as when treating an ethanol/water mixture of more than 80 percent by volume.

As the closest prior art, publication U.S. Pat. No. 4,822,737 is disclosed describing a continuous preparation process for ethanol, in which a concentrated ethanol/water mixture is prepared. Matter is continuously being removed from a fermenter via a solid matter separator to an evaporator, in which ethanol is separated, and the non-volatile matter is introduced into a reverse osmosis unit, from which a separate discharge of water is obtained, and from which the portion containing fermentable sugars is recycled to the fermenter. The process can also be implemented by using two evaporation units, whereby the concentrate obtained from the reverse osmosis unit is introduced into the other evaporation unit for additional removal of ethanol.

### SUMMARY OF THE INVENTION

The method and apparatus according to the invention are characterized by what has been described in the claims.

The invention is based on a method for preparing an ethanol/water mixture, in which fermentable raw materials selected from sugars and raw materials capable of being hydrolyzed into fermentable sugars, and any necessary auxiliary substances are fed into a reactor. Auxiliary substances include, for example, air. The raw materials are fermented in the reactor, and from the fermentation solution thus formed, the ethanol/water mixture is separated in an evaporator, and the non-fermented matter is removed from the reactor. According to the invention, in the method, the reactor fermentation solution is continuously being fed to a first falling-film evaporator during fermentation. The ethanol/water mixture is evaporated in the first falling-film evaporator, and the evaporate obtained from the evaporator is condensed and the condensed ethanol/water mixture is concentrated in a second falling-film evaporator, which is arranged in series with the first falling-film evaporator. Water is removed from the second evaporator as underflow; and the non-volatile matter is removed from the first evaporator and is recycled to the reactor.

The method is specifically based on a) the use of two or more consecutive falling-film evaporators, by means of which it is possible to separate ethanol/water vapor (the evaporate of the second evaporator) and relatively pure water (the underflow of the second evaporator) from the fermentation solution; b) recycling non-volatile matter from the first evaporator to the reactor, whereby the non-fermented sugar in the matter is recycled in a more concentrated form to the reactor thereby accelerating the fermentation for its part; and c) the possibility provided by the use of falling-film evaporators to affect the concentrations and amounts of non-fermented matter, e.g. the concentrations of dry matter and ethanol.

Preferably no fermentable sugars or carbohydrates capable of being hydrolyzed into fermentable sugars are discharged from the process during fermentation.

In a preferred embodiment, the reactor is a fermentation vessel.

In one embodiment of the invention, the water being separated in the second falling-film evaporator is preferably recycled more than once in the second evaporator for minimizing the ethanol concentration of water prior to discharging the water from the evaporator.

In one embodiment of the invention, the fermentation solution is fed from the reactor into a solid matter separator, in which solid matter-containing sludge is separated from the fermentation solution; and from the solid matter separator, the fermentation solution is fed into the first falling-film evaporator. In one embodiment, the solid matter-containing sludge, separated in the solid matter separator, is recycled to the reactor. In one embodiment, the solid matter-containing sludge, separated in the solid matter separator, is removed from the process.

In one embodiment of the invention, the method uses more than two in series connected falling-film evaporators for separating and concentrating the ethanol/water mixture.

In one embodiment of the invention, the ethanol/water mixture is concentrated to a content of 35-65 wt % ethanol, in a preferred embodiment to a content of about 40-50 wt % ethanol.

In the method according to the invention, the condensation can be performed between the evaporators and/or after each evaporator.

The water, free water and dry matter contents, the ethanol content of the so-called residual matter produced in conjunction with the preparation of ethanol, as well as the concentrations of fermentable sugars and sugars capable of being hydrolyzed into fermentable sugars can be affected prior to removal of the residual matter from the process (a) in the fermentation phase by feeding the solution obtained from the solid matter separator into the first falling-film evaporator for recovering the ethanol; (b) in the discharge phase, by feeding the solid matter obtained from the solid matter separator out of the process, when the ethanol content of the solution to be fed from the solid matter separator into the first evaporator has dropped; c) in the fermentation and discharge phase, by feeding water from the second falling-film evaporator back into the evaporator until the ethanol content of the water has decreased.

Furthermore, the invention is based on an apparatus for preparing an ethanol/water mixture, the apparatus including a reactor, e.g. a fermentation vessel, into which the raw materials and any necessary auxiliary substances are fed and in which the raw materials are fermented into a fermentation solution; an evaporator which separates the ethanol/water mixture from the fermentation solution; a conduit for discharging the fermentation solution from the reactor and feeding it into the evaporator, and an exhaust pipe for discharging the non-fermented matter from the reactor. According to the invention, the apparatus includes a first falling-film evaporator for separating the ethanol/water mixture from the fermentation solution and a second falling-film evaporator for concentrating the ethanol/water mixture; and the second falling-film evaporator is arranged in series with the first falling-film evaporator; a condenser for condensing the ethanol/water mixture obtained from the first evaporator prior to feeding it into the second evaporator; an exhaust pipe for discharging the water from the second evaporator as underflow; and a conduit for removing the non-volatile matter from the first evaporator and recycling it to the reactor.

In one embodiment of the invention, the apparatus includes a solid matter separator, which is arranged between the reactor and the first falling-film evaporator, and in which solid matter-containing sludge is separated from the fermentation solution prior to feeding the fermentation solution into the first falling-film evaporator.

In one embodiment of the invention, the apparatus includes a recycling pipe for recycling the solid matter-containing sludge from the solid matter separator to the reactor.

In one embodiment of the invention, the apparatus includes more than two in series connected falling-film evaporators for separating and concentrating the ethanol/water mixture.

The devices of the apparatus are preferably integrated into a whole.

An ethanol/water mixture prepared using the method described above can be used as a constituent of a fuel such as gasoline or diesel, so that the ethanol/water mixture is concentrated to a desired ethanol content, and the concentrated ethanol mixture is mixed with the other fuel constituents to form a predetermined fuel mixture.

In one embodiment, the ethanol/water mixture is further refined by concentrating it to an ethanol content of more than 85 wt % and preferably in Finland more than 99.7 wt %.

The ethanol mixture can be mixed with other fuel constituents in a desired amount and the amount allowed by legislation and product specifications.

The ethanol/water mixture according to the invention can be prepared from wastes of food industry, including biowaste from stores and waste mass from bakeries, milk processing production or potato processing.

Thanks to the invention the output rate of ethanol per fermentation volume can be significantly increased compared to prior-art preparation processes of ethanol. The ethanol/water mixture is led out of the fermentation vessel continuously in conjunction with the fermentation. Thus, a bigger fermentation speed is achieved compared to prior-art methods.

In a smooth, continuous fermentation phase as well as in the discharge phase of the fermentation vessel, the ethanol/water mixture can be concentrated to form a 35-65 wt % ethanol/water mixture, which is a sufficient concentration from the standpoint of further processing, utilization and quality control of ethanol. In a smooth, continuous fermentation phase as well as in the discharge phase of the fermentation vessel, the minor flows in ethanol preparation, such as mash, carbon dioxide and pure water, can be led out of the process as separate flows. The amount by volume of fermentation waste, such as mash, is lesser compared to prior-art processes. Compared to the prior-art inventions, the invention enables one to better affect the quality of the minor flows being produced and the amount and quality of the water obtained as a co-product. For example, carbon dioxide emissions are considerably lower compared to prior-art methods.

Applying the method according to the invention to different raw materials and to relatively small amounts of raw material, e.g. less than 10,000 tons of ethanol per year, is more cost-efficient with respect to the investment and operating costs compared to the prior-art methods. Obtaining the raw material is easy and its price reasonable, because the raw materials used in the method of the invention consist of waste from other industry. In addition, the invention operates in accordance with the principle of sustainable development by utilizing wastes from other industry and by preparing from them desired product for other uses.

Compared to other prior-art methods associated with the enhancement of ethanol preparation, the method according to the invention is simpler and requires less control in use, specifically in the category less than 10,000 tons of ethanol per year. The apparatus of the invention is simple and can be erected in a place where there are wastes to be made use of.

For its part, the invention contributes to the improvement of transportation logistics, because the ethanol/water mixture can be, for example, transported to oil and gasoline terminals as gasoline trucks return.

### LIST OF FIGURES

In the following section, the invention will be described in more detail by means of examples of its embodiments with reference to the accompanying drawings, in which

FIG. 1 represents one apparatus according to the invention;

FIG. 2 represents a second apparatus according to the invention; and

FIG. 3 represents a third apparatus according to the invention.

### DETAILED DESCRIPTION OF THE INVENTION

### Example 1

In this method according to the invention, the output rate of ethanol per fermentation volume can be increased compared to prior-art preparation processes of ethanol. In this method, the ethanol/water mixture is concentrated to a content of about 50 wt % ethanol. Other ethanol/water contents are also possible. Being separate flows, the minor flows produced in the preparation of ethanol can be utilized, and at the same time the amounts and properties of the minor flows can be controlled. In this method, the amounts of waste and waste water are small. In addition, as a co-product, pure water is obtained.

In the method, the falling-film evaporation can be performed at a low pressure and at a low temperature, whereby the ethanol-producing microbe present in the fermentation solution to be fed into the evaporator does not die in the conditions of the evaporator. For example, evaporation using falling-film evaporators at a low pressure of 0.93 bar enables an evaporation temperature of less than 38° C. In that case, e.g. yeast does not die in the conditions of the evaporator prior to being returned to the fermentation vessel. If the ethanol-producing microbe is separated prior to evaporation or if the ethanol-producing microbe functions attached to a carrier, i.e. immobilized, in the fermentation vessel, then the evaporation can be performed at atmospheric pressure, which makes the process simpler. Using a separator prior to evaporation can also be necessary where the fermentation solution contains a considerable amount of solid matter. At the expense of energy economics, falling-film evaporators can be used to concentrate an ethanol/water mixture up to 80 wt % by using recirculation, that is, by returning the condensed evaporate of the evaporators so as to form the feeds for the evaporators.

### Example 2

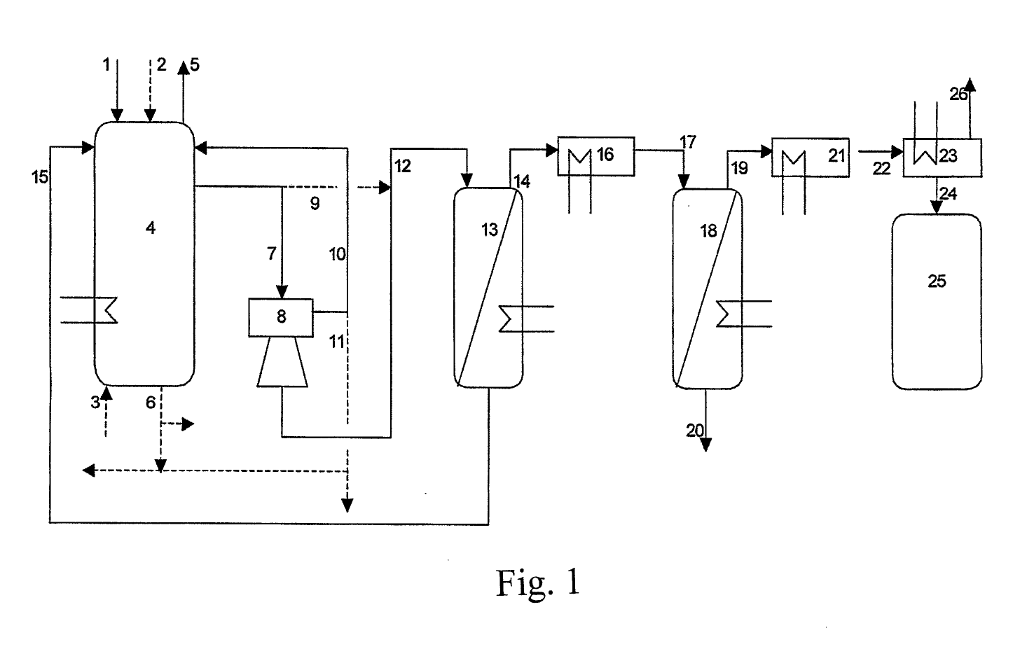

This example describes one method according to the invention and an apparatus for implementing the same. As for the apparatus of this example, reference is made to FIG. 1.

The raw material is fed into a fermentation vessel 4 using a conduit 1. Any necessary auxiliary substances and air can be fed into the fermentation vessel via conduits 2 and 3. If necessary, the content of the fermentation vessel 4, especially the non-fermented matter, can be removed from the process via a conduit 6. The gases produced as a result of ethanol fermentation, including mainly CO₂, are removed from the fermentation vessel via a conduit 5. Depending on the composition of the fermentation solution, as well as on the dry matter content and the amount of the ethanol-producing microbes, the fermentation solution is fed, either using a conduit 7, via a solid matter separator 8, e.g. a decanter centrifuge, into a first falling-film evaporator 13 using a conduit 12, or directly from the fermentation vessel 4 into the first falling-film evaporator 13 using a conduit 9. From the solid matter separator 8, the solid matter-rich sludge is fed back into the fermentation vessel 4 using a conduit 10. If necessary, the solid matter-rich sludge in the solid matter separator 8 can be led out of the process via a conduit 11. From the first falling-film evaporator 13, the evaporate is fed via a conduit 14 into a condenser 16, from which the liquefied flow is introduced via a conduit 17 into a second falling-film evaporator 18. From the first falling-film evaporator 13, the nonvolatile matter is fed into the fermentation vessel via a conduit 15. The underflow of the second falling-film evaporator 18, which is mainly water, is removed using a conduit 20. The evaporate of the second falling-film evaporator 18 is fed via a conduit 19 into a condenser 21 and from there via a conduit 22 into a final condenser 23, from which the completely condensed ethanol/water mixture is fed via a conduit 24 into a storage tank 25 for ethanol/water mixture. The off-gas of the final condenser is removed from the process via a conduit 26. It is further possible to use in the process heat exchangers between flows 12 and 14, and 17 and 19. The final condensation 23 is a relatively small flow, and it is possible to use in it a heat exchanger with cold matter circulation condensing water to about 200° C. In the embodiment of FIG. 1 there is one fermentation vessel, but there can be more than one fermentation vessels in series or in parallel. In the embodiment of the figure, there are two falling-film evaporators 13 and 18 in series, but there can be more than two falling-film evaporators in series.

The fermentation vessel 4 has three different run phases: 1) filling 2) fermentation and addition of feed within the limits of the level and 3) discharging of the fermentation vessel.

The filling phase starts with the feeding of the raw material and any necessary auxiliary substances via conduits 1 and 2 into the fermentation vessel 4. If necessary, to increase the mass of the ethanol-producing microbe, sterilized air or oxygen can be fed into the fermentation vessel via a conduit 3. In the filling phase, conduits 1, 9, and 6 are closed. In the fermentation phase, feed is added into the fermentation vessel 4 via a conduit 1 within the limits of the level of the fermentation vessel, the ethanol content of conduit 22, and the sugar content of the fermentable raw material of the fermentation vessel, as well as within the limits of the carbohydrate content of the raw material capable of being hydrolyzed into fermentable sugars. If the solid matter of the fermentation solution causes a risk of contamination to the falling-film evaporator, the fermentation solution is fed via a conduit 7 into the solid matter separator 8. If the contamination caused by the solid matter of the fermentation solution is allowed, the fermentation solution is fed via a conduit 9 directly into the falling-film evaporator 13. In that case, ethanol can be obtained from the fermentation solution into the storage tank 25, and at the same time, water is discharged from the fermentation solution via conduit 20. The evaporation in the falling-film evaporators can be performed at a normal pressure, whereby the temperature of the solution being discharged is about 80-85° C., or at a low pressure, e.g. at a low pressure of 0.93 bar, whereby the temperature of the solution being discharged is about 36° C. In a sufficient low pressure evaporation, e.g. at a low pressure of 0.93 bar and at a temperature of 3β° C., the ethanol-producing microbe need not necessarily be separated from the solution to be evaporated, but the solution of the fermentation vessel 4 can be fed, without the microbes dying, into the falling-film evaporator 13 via a conduit 9. Furthermore, the separation is not necessary if the microbe making ethanol, e.g. yeast, is immobilized. Using a low pressure in the falling-film evaporators 13 and 18 reduces the need for the necessary evaporation energy, but increases the volume of the condensers 16, 21 and 23 and weakens the heat transfer of the condensers.

The conduit 12 to the falling-film evaporator 13 can be closed part of the time in the fermentation phase within the limits of the level of the fermentation vessel 4 and within the limits of the flow of the conduit 1. On an average, about 40 wt % ethanol/water mixture is produced into the product storage tank 25, and it is a sufficient content bearing in mind the further processing.

In the discharge phase, the non-fermented matter and/or water formed therein, which weaken the smooth, continuous operation, are discharged from the fermentation vessel 4. The discharge phase is performed when the non-fermented matter weakens the fermentation result, and when the fermentable sugars and carbohydrates capable of being hydrolyzed into fermentable sugars are fermented as exhaustively as possible in the fermentation vessel. In the discharge phase, no more raw materials can be fed into the fermentation vessel within the limits of the level. In that case, the fermentation solution is fed via the conduit 7 into the solid matter separator 8 and from there via the conduit 12 to the falling-film evaporator 13, or directly to the falling-film evaporator 13. When the ethanol content of the fermentation solution is sufficiently low, e.g. 0.5 wt %, the conduit 9 and conduit 12 will be closed, and the sludge is removed from the process via a conduit 11, and the non-fermented matter in the fermentation vessel is removed from the process via a conduit 6.

In condensers 16 and 21 it is possible to use external cooling. In condenser 23, one has to provide a sufficiently low temperature for the vapor in order for the discharge of ethanol via a conduit 26 to be as small as possible. In addition, in the feed, fermentation and discharge phases, the temperature of the fermentation vessel can be adjusted, in other words, cooled, as a main rule.

### Example 3

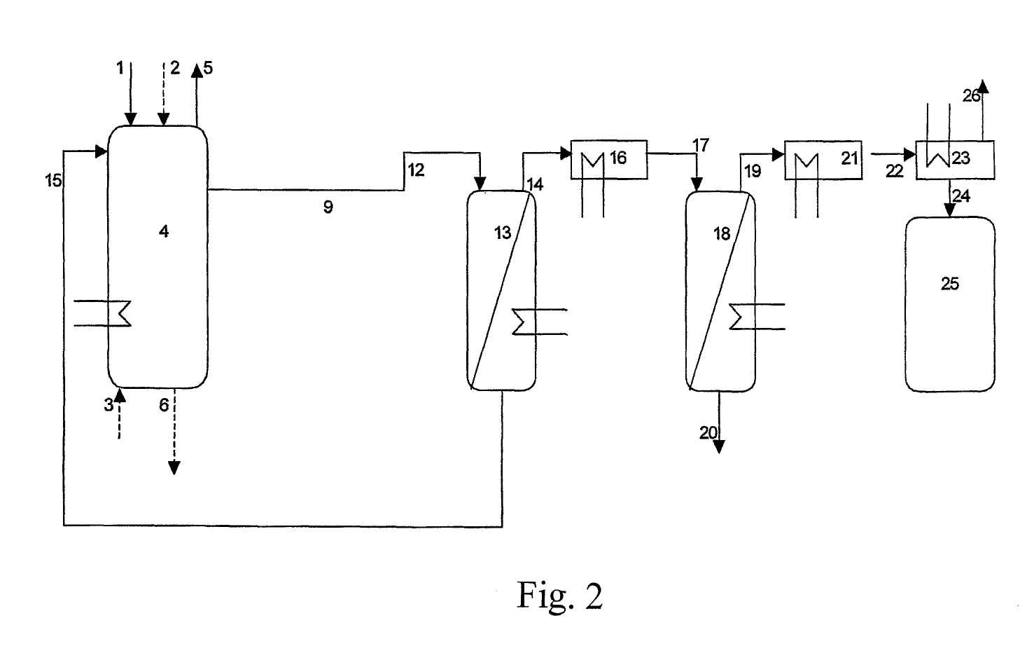

This example shows the use of whey, a co-product of the cheese making process, as a raw material in the process in accordance of the invention; reference is made to FIG. 2.

A typical whey formed as a co-product in the cheese making process contains lactose 4.8 wt %, which, as such, acts as a substrate for the ethanol making microbe; and e.g. genetically modified yeasts can act as the ethanol making microbe. Typically, whey contains solid matter less than 3 wt %. In this example, the ethanol producing microbe has been immobilized in the fermentation vessel by means of prior-art technology, whereby the falling-film evaporators 13 and 18 and the condensers 16, 21 and 23 act at air pressure in a temperature ranging from 80 to 90° C. The non-fermented matter can be used as juice fodder, the carbohydrate content of which is lower than previously, but the protein content considerably higher than previously. In the fermentation phase, with a feed flow 1 of 0.9 kg/s, the outlet flow 24 is 0.054 kg/s and the ethanol content of the outlet flow is 40 wt %. In that case, the flow 20 is 0.306 kg/s, and consists of nearly pure water. The amount of the flow 9 is 1.44 kg/s and its ethanol content is 1.5 wt %, and sugar content 1.8 wt %. The reflux 15 of the non-volatile matter is 1.08 kg/s, and it does not contain ethanol, and its sugar content is 2.4 wt %. In other words, in this example, the fermentation solution is cumulated into the fermentation vessel at a rate of 0.54 kg/s, that is, the low sugar content of whey results in a semi-continuous process with the method according to the invention, and it is possible to feed whey into the process within the limits of the level of the fermentation vessel 4.

### Example 4

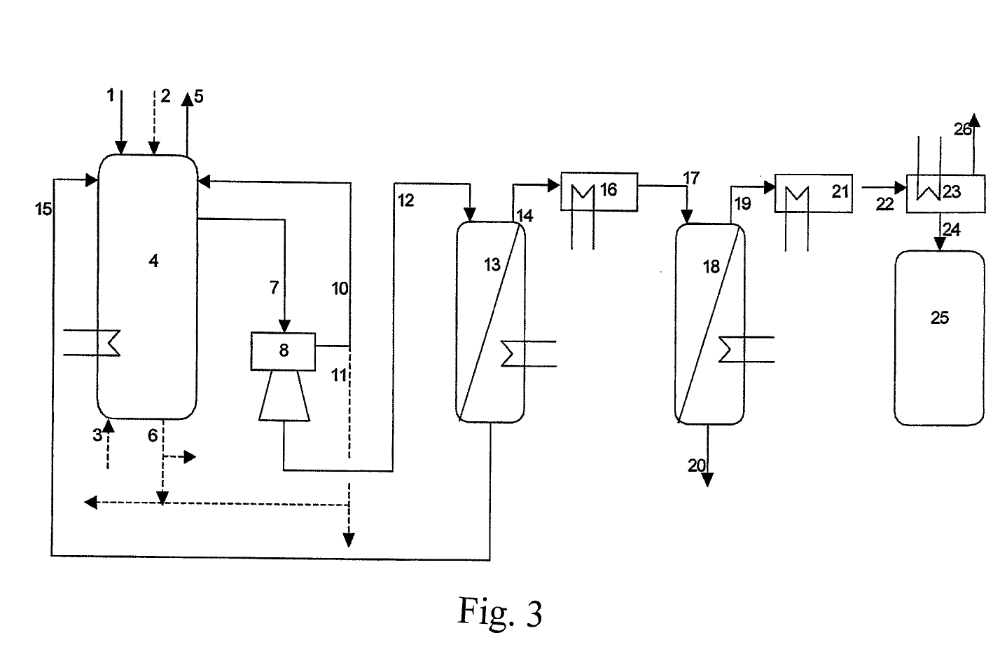

This example shows the use of waste dough as the raw material of the apparatus according to the invention; reference is made to FIG. 3.

Waste dough typically contains about 50-60% of dry matter, of which 60-80 wt % is starch originating from grain, and about 5 wt % consists of different sugars. The waste dough has been diluted with water, and the starch contained in it has been hydrolyzed into sugars prior to feeding it into the process according to the invention. In this example, the dry matter and the ethanol producing microbe, which is yeast, are separated from the fermentation solution prior to feeding into the falling-film evaporators 13 and 18, whereby the evaporators and the condensers 16, 21 and 23 operate at a normal air pressure in a temperature ranging from 80 to 90° C. The non-fermented matter can be used as fodder. In the fermentation phase, when the feed flow 1 is 0.1 kg/s, the outlet flow 24 is 0.0125 kg/s, and the ethanol content of the outlet flow is 40 wt %. In that case, the flow 20 is 0.0708 kg/s and it is nearly pure water. The amount of the flow 12 is 0.166 kg/s and its ethanol content is 3.0 wt % and sugar content 4.0 wt %. The reflux 15 of the nonvolatile matter is 0.083 kg/s, and it does not contain ethanol, and its sugar content is 9.0 wt %. Non-fermented matter is formed at a rate of 0.0166 kg/s, which matter is removed during the discharge phases. In this example, no fermentation solution is cumulated into the fermentation vessel, and the embodiment according to the invention is continuously operating.

In applying the method according to this invention, it is possible to prepare from the various minor and waste flows produced in food processing industry, agriculture and other industry a 35-65 wt %, preferably a 40-50 wt % ethanol/water mixture to be further processed so as to have the desired ethanol content, for example, to be used as a fuel for vehicles, or as a solvent for various industry processes using ethanol mixtures, or as other uses of ethanol or an ethanol mixture. The non-fermented matter/waste in the reactor or fermentation vessel that is produced in the method according to the invention can be utilized as animal fodder or as a soil conditioner.

The invention is not limited merely to the examples of its embodiments referred to above; instead many variations are possible within the scope of the inventive idea defined by the claims.

1.-17. (canceled)

18. A method for preparing an ethanol/water mixture, comprising:
feeding fermentable raw materials, selected from sugars and raw materials capable of being hydrolyzed into fermentable sugars, and any necessary auxiliary substances into a reactor;
fermenting the raw materials in the reactor to form a reactor fermentation solution;
separating the ethanol/water mixture from the reactor fermentation solution in a first falling-film evaporator to create an evaporate, the reactor fermentation solution continuously being fed into the first falling-film evaporator during fermentation;
condensing the evaporate from the first falling-film evaporator and concentrating the condensed evaporate in a second falling-film evaporator arranged in series with the first falling-film evaporator;
removing water from the second falling-film evaporator;
removing any non-volatile matter from the first falling-film evaporator and recycling it to the reactor; and
removing any non-fermented matter from the reactor.

19. The method as defined in claim 18, characterized in that the reactor is a fermentation vessel.

20. The method as defined in claim 18, characterized in that the water being separated in the second falling-film evaporator is recycled in the evaporator to minimize the ethanol concentration of water prior to removing the water from the evaporator.

21. The method as defined in claim 18, further comprising:
feeding the fermentation solution from the reactor into a solid matter separator;
separating any solid matter-containing sludge from the fermentation solution; and
feeding the fermentation solution from the solid matter separator into the first falling-film evaporator.

22. The method as defined in claim 21, characterized in that the solid matter-containing sludge separated by the solid matter separator is recycled to the reactor.

23. The method as defined in claim 21, characterized in that the solid matter-containing sludge separated by the solid matter separator is removed from the process.

24. The method as defined in claim 18, characterized in that the method uses more than two falling-film evaporators connected in series for separating and concentrating the ethanol/water mixture.

25. The method as defined in claim 18, characterized in that the ethanol/water mixture is concentrated to a concentration of about 35-65 wt % ethanol.

26. The method as defined in claim 25, characterized in that the ethanol/water mixture is concentrated to a concentration of about 40-50 wt % ethanol.

27. The method as defined in claim 18, characterized in that the raw material is fed via a conduit into the fermentation vessel; any necessary auxiliary substances are fed into the fermentation vessel via conduits; when emptying the fermentation vessel, the non-fermented matter is removed via a conduit; the gases produced in the fermentation vessel as a result of ethanol fermentation are removed from the fermentation vessel using a conduit; depending on the composition of the fermentation solution, the fermentation solution is fed either via a conduit through the solid matter separator into the first falling-film evaporator or directly from the fermentation vessel via a conduit into the first falling-film evaporator; from the solid matter separator, the solid matter-rich sludge is recycled to the fermentation vessel using a conduit, or the solid matter-rich sludge in the solid matter separator is removed from the process using a conduit; from the first falling-film evaporator, the evaporate is fed via a conduit into a condenser, from which the liquefied flow is fed via a conduit into a second falling-film evaporator connected in series; from the first falling-film evaporator, the non-volatile matter is fed via a conduit into a fermentation vessel; the underflow of the second falling-film evaporator, consisting mainly of water, is removed via a conduit; the evaporate of the second falling-film evaporator is fed via a conduit into a condenser and from there via a conduit into a final condenser, from which the condensed ethanol/water mixture is fed via a conduit into a storage tank of the ethanol/water mixture, and the off-gas of the final condenser is discharged from the process via a conduit.

28. An apparatus for preparing an ethanol/water mixture, the apparatus comprising:
a reactor, into which raw materials and any necessary auxiliary substances are fed and in which the raw materials are fermented into a fermentation solution;
an evaporator in which ethanol/water mixture is separated from the fermentation solution, the evaporator including a first falling-film evaporator for separating the ethanol/water mixture from the fermentation solution and a second falling-film evaporator for concentrating the ethanol/water mixture, the second falling-film evaporator arranged in series with the first falling-film evaporator;
a conduit for discharging the fermentation solution from the reactor and introducing the fermentation solution into the evaporator;
a first exhaust pipe for discharging the non-fermented matter from the reactor;
a condenser for condensing the ethanol/water mixture obtained from the first evaporator prior to feeding into the second evaporator;
a second exhaust pipe for discharging water from the second evaporator as underflow; and
a conduit for discharging non-volatile matter from the first evaporator and for recycling it to the reactor.

29. The apparatus as defined in claim 28, characterized in that the reactor is a fermentation vessel.

30. The apparatus as defined in claim 28, further comprising a solid matter separator which is arranged between the reactor and the first falling-film evaporator, in which any solid matter-containing sludge is separated from the fermentation solution.

31. The apparatus as defined in claim 30, further comprising a recycling pipe for recycling the solid matter-containing sludge from the solid matter separator to the reactor.

32. The apparatus as defined in claim 28, further comprising more than two series-connected falling-film evaporators for separating and concentrating the ethanol/water mixture.

33. Use of an ethanol/water mixture prepared according to claim 18 as a constituent of fuel, characterized in that the ethanol/water mixture is concentrated to the desired ethanol content and the concentrated ethanol mixture is mixed with other fuel constituents to form a predetermined fuel mixture.

34. Use of an ethanol/water mixture as a constituent of fuel, characterized in that the ethanol/water mixture is concentrated to an ethanol mixture of more than 85 w %.
